(19) United States
(12) Patent Application Publication
(10) Pub. No.: US 2005/0165090 A1
(43) Pub. Date: Jul. 28, 2005

(54) Polyphenols as an intravesical agent for the prevention of transitional cell tumor implantation

(76) Inventors: J. Karl Kemberling, Duluth, MN (US); Steven H. Selman, Toledo, OH (US)

Correspondence Address: EMCH, SCHAFFER, SCHAUB & PORCELLO CO, P O BOX 916, ONE SEAGATE SUITE 1980, TOLEDO, OH, 43697, US

(21) Appl. No.: 11/040,908

(22) Filed: Jan. 21, 2005

Related U.S. Application Data
(60) Provisional application No. 60/538,699, filed on Jan. 23, 2004.

(51) Int. Cl. A61K 31/353

(52) U.S. Cl. 514/456

(57) ABSTRACT

A method and pharmaceutical composition for treating the bladder cancer in a mammal includes intravesically administrating to a mammal in need of bladder cancer treatment a therapeutically effective amount of a composition comprising at least one polyphenol and in particular EGCG, within the mammal's bladder.

### CROSS REFERENCE TO RELATED APPLICATIONS

This application claims the benefit of U.S. provisional patent application Ser. No. 60/538,699 filed Jan. 23, 2004.

### FIELD OF INVENTION

The present invention relates to a method and a pharmaceutical composition for treating the bladder cancer in a mammal. In particular, at least one catechin, such as EGCG, is intravesically administrated to a mammal in need of bladder cancer treatment.

### BACKGROUND OF THE INVENTION

Bladder cancer is a common malignancy. The majority of bladder cancers are superficial; however, there is a high rate of recurrence and 13.3% of cases will progress to muscular invasion. The risk of recurrence after transurethral resection is more than 60% in 7 years. Tumor implantation at the time of tumor resection may account in part for the high rate of tumor recurrence. A number of intravesical agents have been utilized in an attempt to decrease the rate of recurrence. Green tea extracts have been studied extensively in several tumor modes, but not for their potential as intravesical chemotherapeutic agents.

Green tea is one of the world's most popular beverages recently attracting attention as a natural health-promoting agent. The emperor of China, Shen Nung, is credited with first describing the therapeutic effects of tea in 2737 B.C. Although the epidemiologic data is conflicting, reports have suggested an inverse relationship between green tea consumption and cancer incidence. A study by Bianchi et al. suggest that those consuming greater than 5 cups of tea per day may have a decreased incidence of bladder cancer. While most Western populations do not consume large amounts of tea, this quantity is not uncommon in the Asian subcontinent.

Green tea is made from the leaves of Camellia Sinensis and contains higher concentrations of polyphenolic catechins than black or Oolong tea. Epigallocatechin-3-gallate (EGCG) is green tea's most abundant catechin and has been shown to exhibit significant anti-oxidant, anticarcinogenic and antimicrobial properties. EGCG is water-soluble with a molecular weight of 458.4. By virtue of its structure and stereochemistry, polyphenolic rings carry numerous hydroxyl groups making it an excellent scavenger of free radicals.

In vivo studies reveal that EGCG is bioavailable in both the blood and urine in low micromolar concentrations after the consumption of green tea. To the best of our knowledge, the use of catechins as an intravesical chemotherapy has never been studied or evaluated.

A number of antineoplastic properties of EGCG have been described including inhibition of tumor initiation and promotion, induction of apoptosis, and cell cycle arrest. Morre et al. have shown preferential inhibition of cancer cells treated by EGCG related to suppression of NADH oxidase activity. EGCG blocks the activation of transcription factor nuclear factor-κB (NF-κB) which induces NO synthase. Nitric oxide (NO) is known to play a role in both carcinogenesis and inflammation. EGCG inhibits activator protein-1 (AP-1) transcriptional activity and DNA binding activity. AP-1 is believed to be a tumor promoter. Inhibition of angiogenesis in several tumor models has also been demonstrated.

In spite of the above studies, the use of catechins, and in particular EGCG, as a therapeutic agent used in intravesically for the prevention of transitional cell tumor implantation has, to the best of the inventors' knowledge, never been reported before.

There remains a need for the prevention of cancer, and in particular, tumor implantation in the bladder.

Further, there is a need for an intravesical agent for use in the bladder which does not have the adverse effects generally associated with conventional chemotherapeutic agents. It is to be understood that none of the above mentioned research, which is not to be construed as an admission that any of the above research is prior art, has suggested the present method in which catechins, and in particular EGCG, is instilled intravesically to inhibit the growth of cancer cells.

Further, none of the research evaluated the efficacy of catechins, and in particular EGCG, as intravesical chemotherapeutic agent. In contrast, the inventors herein have identified a method of preventing transitional cell tumor implantation.

Further, while the focus of other studies and patents including U.S. Pat. Nos. 6,410,061 B1, 6,410,052 B1, 6,428,818 B1 and 6,652,890 B2 describe methods for treating cancer or solid tumors with EGCG, the efficacy of catechins, and in particular EGCG, as a single therapeutic agent which can be used intravesically for the prevention of tumor cell implantation was not known until the present invention.

### SUMMARY OF THE INVENTION

The invention described herein encompasses, in part, a method of treating bladder cancer comprising administrating a therapeutically effective amount of a polyphenol such as EGCG to a mammal in need of such therapy.

In certain environments, the invention further relates to a method of treating bladder cancer comprising intravesically administrating a therapeutic amount of a polyphenol such as EGCG.

In a specific embodiment, the method comprises intravesically administrating the polyphenol, and in particular EGCG, in a sustained release manner. It is within the contemplated scope of the present invention that the polyphenols, and in particular EGCG, can be configured to be part of a sustained release preparation for placement in the bladder through the urethra. It is also contemplated that such sustained release preparations can be bio-erodable such that during or after the release of the drug there is no blockage of the urinary tract while the sustained release preparation is within the bladder.

It is also within the contemplative scope of the present invention that the polyphenols, and in particular EGCG, can be used alone or in combination with other known therapeutic agents and methods of treatment. The intravesical administration of polyphenols, and in particular EGCG, can be used before during and after administration of other therapeutic agents and methods including surgical resection, radiation and chemotherapy.

Other objects and advantages of the present invention will become apparent to those skilled in the art upon a review of the following detailed description of the preferred embodiments and the accompanying drawings.

### BRIEF DESCRIPTION OF THE FIGURES

FIG. 1 is a graph showing the in vivo studies of the AY-27 cell line exhibiting a time-dosed related response to treatment with various concentrations of EGCG.

FIG. 2 is a table showing in vivo study using intravesicular installation of 200 μM EGCG solution for two hours in the Fisher 334 rat model.

FIG. 3 is an illustration (control) showing a bladder with papillary tumor growth (arrow).

FIG. 4 is an illustration showing a bladder, treated with 200 μM of Polyphenon E, having normal bladder mucosa.

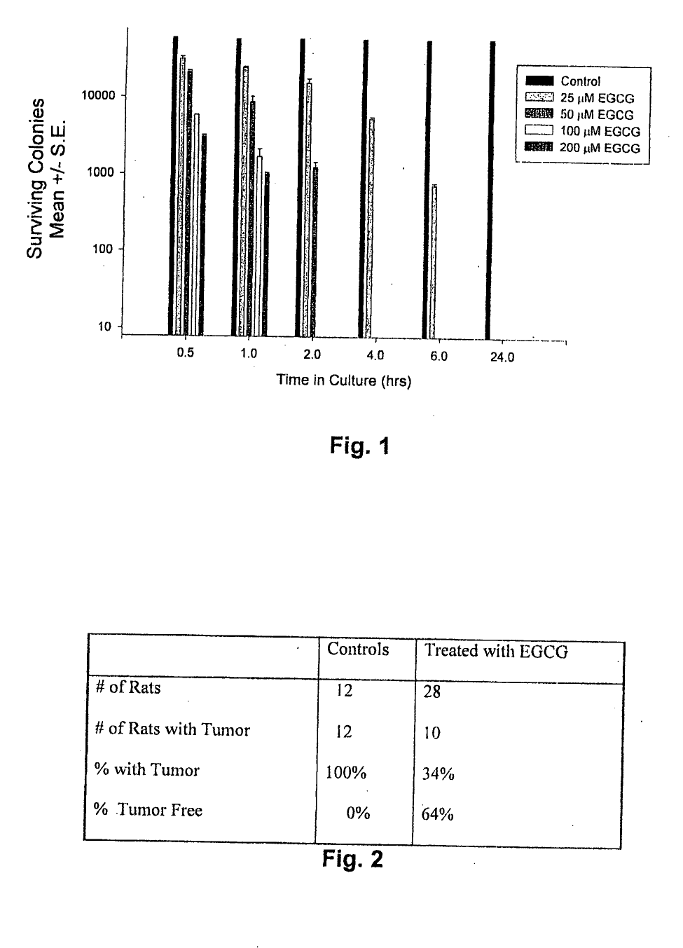

### DETAILED DESCRIPTION OF THE INVENTION

This invention is a method for treating bladder cancer in a mammal which comprises administering to a mammal in need of bladder cancer treatment a therapeutically effective amount of a composition comprising at least one polyphenol wherein the composition is instilled intravesically within the patient's bladder. Preferably, the polyphenol is represented by the formula. 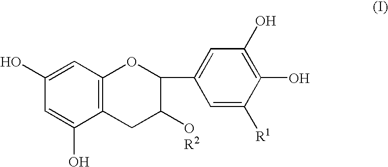
wherein R¹ represents H or OH and R² represents H or

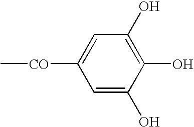

Typically, the polyphenol comprises at least one catechin. Preferably, the polyphenol comprises epigallocatechin-gallate (EGCG) or polyphenon E. In one embodiment, the composition comprises the polyphenol in combination with at least one plant metabolite. The plant metabolite is selected from the group consisting of polyphenols, complex polyphenols, bioflavonoids, flavonols; bioflavanols, flavones and catchins. A preferred plant metabolite comprises flavonol.

In a particular preferred embodiment, the present invention relates to the treatment of humans comprising intravesically administrating a therapeutic amount of catechins, and in particular EGCG.

In the preferred embodiment the patient has cancer and is undergoing treatment of cancer which includes intravesically administrating a therapeutic amount of catechins, and in particular EGCG. In another embodiment, the patient is treated to prevent the implantation of any tumor cells following a resection of a solid tumor from the patient's bladder. It is to be understood that according to the method of the present invention, the preparation disperses and dissolves or disintegrates in a time released manner so as to provide a controlled release of the catechins, and in particular EGCG, for an extended period of time. It is to be understood that the carrier materials can be any pharmaceutically acceptable carrier which is instilled into the bladder by any number of methods known in the art for forming such materials which contain medicaments. The devices or carriers containing the drug can be inserted into the bladder through the urethra using a catheter. The dosages administered would depend on the protocol required for the treatment of the particular condition and to the patient.

Tea catechins may be for example, (−) -epicatechin, (−) -epicatechin gallate, (−) -epigallocatechin, (−) -epigallocatchin gallate, (−)-gallocatechin. These catechins may be used singly or a combination of two or more compounds. Among these catechins, compounds containing (−)-epicatechin gallate and/or (−)-epigallocatechin gallate are preferable. Other tea catechins which contain (−)-epigallocatechin gallate as main components include “Polyphenon 100, composition: (+)-gallocatechin 1.44%, (−)-epicatchin 5.81%, (−)-epigallocatechin 17.57%, (−)-epicatechin gallate 12.51%, (−)-epigallocatechin gallate 53.9% and “Polyphenon E”, composition: (−)epicatechin 10.8%, (−)-epigallocatechin 9.2%, (−)-epicatechin gallate 6.5%, (−)-epigallocatechin gallate 54.8%, (−)-gallocatechin gallate 4.0%).

Examples of plant metabolites follow. Grape seed extract is a bioflanol known as OPC. Other common extracts are from pine bark and peanuts.

Bioflavanols are a group of anti oxidants belonging to the bioflavonoid family known as “Vitamin P”. They are known as OPC, proanthocyanidins, procyanidins, leucocyanidins, leukocyanidol and pyconogenols. These are free radical scavengers and vitamin C enhancers. In France where research first began, OPC was the abbreviated name for “oligomeres procyanidoliques”.

By way of illustration the following examples are given showing the efficacy of the present invention, however, these are for purposes of illustration and are not intended in any way to limit the scope of the invention.

### EXAMPLE 1

Cell Culture: The AY-27 rat transitional cell line was grown in RPMI 1640 media supplemented with 10% fetal bovine serum. The L1210 Mouse Leukemia cell line was grown in Fisher's medium supplemented with 10% equine serum and used for DNA ladder assay studies. 6.0×10⁴ AY-27 cells were placed onto 100 mm culture dishes and incubated for 24 hours. This cell concentration was representative of in vivo cell tumor instillation burden. Increasing concentrations of EGCG (25-300 μM) in RPMI complete media were added to cell plates for 30 minutes. 1,2,4,6 and 24 hour periods after which culture media was removed and replaced with RPMI without EGCG. Surviving cells were incubated for 3 days to allow for colony formation, then rinsed with 10% saline, fixed with 100% methanol and the experiments were repeated in quadruplicate to determine effective treatment concentrations.

DNA ladder assay: The AY-27 cells were grown to approximately 70% confluency in 75 cm flasks containing RPMI complete medium before treatment with EGCG (100,200 μM) for 24-48 hours. The L1210 Mouse Leukemia cell line was also grown in Fisher's Media Complete suspension and treated with EGCG for 24 hours. After treatment with EGCG, cell lines were evaluated with DNA ladder assay. A 2% agarose gel was used with an ethidium bromide stain and run for 30 minutes using a 1-kbp reference ladder.

Results Clonal Growth Assays: Following exposure to EGCG, the survival of AY-27 cells revealed a time-dose dependent response. At 2 hours of exposure with EGCG concentrations of 100 μM or greater, 100% cell lethality was observed.

Light Microscopy: Both the AY-27 transitional cancer cells and the L1210 cell lines treated for 2 hours show morphologic changes including cellular shrinkage, pyknosis and surface blebbing.

DNA Ladder Assays: The L1210 mouse leukemia cell line exhibited a distinct, vibrant banding pattern suggestive of apoptosis; however, the AY-27 cells treated for 24 hours with EGCG (100 and 200 μM) showed a weak banding pattern.

### EXAMPLE 2

In Vivo Study: Fisher 344 rats were maintained under guidelines of the Medical College of Ohio Institutional Animal Care and Use Committee. A combination ketamine (80 mg/kg)/xylazine(12 mg/kg) intraperitoneal injection was used to anesthetize the rats prior to surgery. The abdomen was shaved and prepped with an iodine solution under sterile conditions. Oxytetracycline 20 mg was injected subcutaneously for antibacterial prophylaxis. Under 4× microscopic magnification, a 1.5 cm midline incision exposed the bladder. A sterile, 22 gauge Angiocath™ cannula was then advanced transurethrally. A blunt-tipped 24 gauge copper wire was then advanced through the catheter and a 6 m V current was used to cauterize the bladder wall creating a small, white puckered area. The skin and muscular layers of the abdominal wall were closed with a running 4-0 silk suture.

AY-27 cells suspended in 0.25 cc of serum free RPMI-1640 medium were instilled transurethrally into 40 rats. The catheters were then capped and the tumor cells were allowed to dwell inside the bladder for 30 minutes. After tumor cell instillation, twelve rats (Group I) had two, separately hourly instillations of culture medium. Twenty-eight rats (Group II) were treated with a freshly prepared solution of 0.25 cc, 200 μM EGCG solution in RPLMI-1640 medium for 2 separate dwell times of one hour.

At the end of two hours the catheters were drained. All rats survived and were transferred to the animal care facility for recovery. The rats were euthanized three weeks later. Bladders were removed and placed in 10% phosphate buffered formalin solution and allowed to fix for several hours. Bladders were hemisected with a razor, examined under 4× magnification and sent for histopathologic sectioning.

Statistical analysis was performed using Sigma Stat 2.03 software.

Results

In Vivo Study: Group I controls (n=12) demonstrated 100% tumor growth rate. Group II (n=28), EGCG treated rats had a tumor growth rate of 36% (10 of 28 rats). Statistical significance was achieved with a p value of 0.001 using the Fisher exact test.

Numerous theories have evolved in an attempt to explain the action of EGCG, but our results for the AY-27 cell line do not indicate that apoptosis is the primary mechanism of action.

No cellular survival was seen after two hours of treatment at all concentrations of EGCG. The L1210 leukemia cell line is well known for its ability to undergo apoptosis when treated with various chemical solvents. In the example shown herein, it showed prominent blebbing, cellular shrinkage, and nuclear condensation, suggestive of apoptosis. The AY-27 cells only revealed a weak banding pattern on DNA ladder assay, suggesting that other mechanisms may be involved in their death.

The green tea extract, epigallocatechin-gallate (EGCG), inhibits cellular growth of the AY-27 rat transitional cancer cell line. Instillation of intravesical EGCG at 200 μM for 2 hours appear to significantly reduce the incidence of bladder tumor implantation in the Fisher 344 rat model. To our knowledge, this is the first study evaluating the potential of EGCG as an intravesical chemotherapeutic agent.

### EXAMPLE 3

Another study was done according to the procedure of Example 1 and 2 with Polyphenon E.

In Vivo Study: Fisher 344 rats were maintained under guidelines of the Medical College of Ohio Institutional Animal Care and Use Committee. A combination ketamine (80 mg/kg)/xylazine(12 mg/kg) intraperitoneal injection was used to anesthetize the rats prior to surgery. The abdomen was shaved and prepped with an iodine solution under sterile conditions. Oxytetracycline 20 mg was injected subcutaneously for antibacterial prophylaxis. Under 4× microscopic magnification, a 1.5 cm midline incision exposed the bladder. A sterile, 22 gauge Angiocath™ cannula was then advanced transurethrally. A blunt-tipped 24 gauge copper wire was then advanced through the catheter and a 6 m V current was used to cauterize the bladder wall creating a small, white puckered area. The skin and muscular layers of the abdominal wall were closed with a running 4-0 silk suture.

AY-27 cells suspended in 0.25 cc of serum free RPMI-1640 medium were instilled transurethrally into 10 rats. The catheters were then capped and the tumor cells were allowed to dwell inside the bladder for 30 minutes. After tumor cell instillation, five rats (Group I) had two, separately 30 minute instillations of culture medium. Five rats (Group II) were treated with a freshly prepared solution of 0.5 cc, 200 μM Polyphenon E solution in RPLMI-1640 medium for 2 separate dwell times of 30 minutes.

At the end of one hour the catheters were drained. All rats survived and were transferred to the animal care facility for recovery. The rats were euthanized three weeks later. Bladders were removed and placed in 10% phosphate buffered formalin solution and allowed to fix for several hours. Bladders were hemisected with a razor, examined under 4× magnification and sent for histopathologic sectioning.

Statistical analysis was performed using Sigma Stat 2.03 software.

Results

In Vivo Study: Group I controls (n=5) demonstrated 100% tumor growth rate. Group II (n=5), Polyphenon E treated rats had a tumor growth rate of 20%. Statistical significance was achieved with a p value of 0.001 using the Fisher exact test.

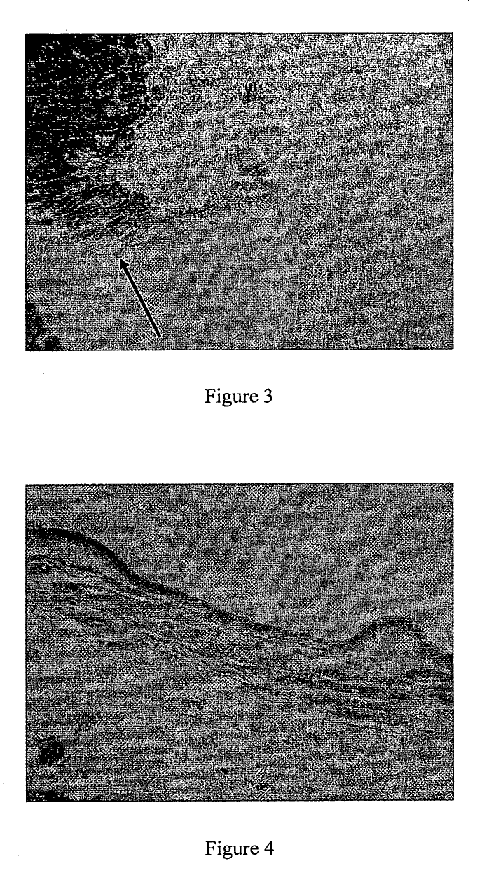

FIG. 3 is an illustration (control) showing a bladder with papillary tumor growth (arrow). FIG. 4 is an illustration showing a bladder, treated with 200 μM of Polyphenon E, having normal bladder mucosa.

In another aspect, the present invention encompasses a kit for the treatment of bladder cancer where the kit can include a pharmaceutical preparation containing at least one catechin for installation in the bladder, at least one catheter and other appurtenances required for instillation into the bladder, and instructions for installing the catheter in the bladder and instilling the at least one catechin pharmaceutical preparation.

The present invention is not to be limiting in scope by the specific embodiments described herein. Indeed, various modifications of the invention in addition to those described will be, apparent to those skilled in the art from the foregoing description and accompanying figures. Such modifications are intended to fall within the scope of the appended claims.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

1. A method for treating the bladder cancer in a mammal which comprises administrating to a mammal in need of bladder cancer treatment a therapeutically effective amount of a composition comprising at least one polyphenol wherein the composition is instilled intravesically within the patient's bladder.

2. The method of claim 1 wherein the polyphenol is represented by formula (I): 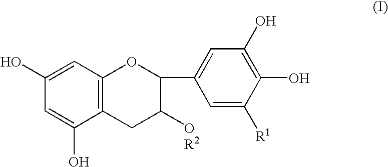
wherein R¹ represents H or OH and R² represents H or

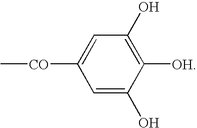

3. The method of claim 1 wherein the polyphenol comprises at least one catechin.

4. The method according to claim 1 wherein the polyphenol comprises epigallocatechin-gallate (EGCG).

5. The method according to claim 1 wherein the polyphenol comprises polyphenon E.

6. The method according to claim 1 wherein the composition comprises a mixture of polyphenols.

7. The method of claim 1 wherein the composition is a mixture of catechins.

8. The method according to claim 1 wherein the composition comprises the polyphenol in combination with at least one plant metabolite.

9. The method of claim 8 wherein the plant metabolite is selected from the group consisting of polyphenols, complex polyphenols, bioflavonoids, flavonols, bioflavanols, flavones and catchins.

10. The method of claim 8 wherein the plant metabolite comprises flavonol.

11. The method of claim 1 in which the composition is instilled intravesically in the patient's bladder before, during or after administration of other chemotherapeutic agents.

12. The method of claim 1 wherein the mammal is a human.

13. A pharmaceutical composition comprising an effective amount of at least one polyphenol to inhibit and/or treat bladder cancer in a mammal wherein the pharmaceutical composition comprises the at least one polyphenol along with a pharmaceutically acceptable carrier for instilling the pharmaceutical composition within the patient's bladder.

14. The pharmaceutical composition of claim 13 wherein the polyphenol is represented by formula (I): 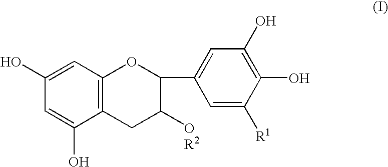
wherein R¹ represents H or OH and R² represents H or

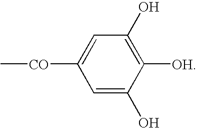

15. The pharmaceutical composition of claim 13 wherein the polyphenol comprises at least one catechin.

16. The pharmaceutical composition of claim 13 wherein the polyphenol comprises epigallocatechin-gallate (EGCG).

17. The pharmaceutical composition of claim 13 wherein the polyphenol comprises polyphenon E.

18. The pharmaceutical composition of claim 13 wherein the composition comprises a mixture of polyphenols.

19. The pharmaceutical composition of claim 13 wherein the composition comprises a mixture of catechins.

20. The pharmaceutical composition according to claim 13 wherein the composition comprises the polyphenol in combination with at least one plant metabolite.

21. The pharmaceutical composition according to claim 20 wherein the plant metabolite is selected from the group consisting of polyphenols, complex polyphenols, bioflavonoids, flavonols, bioflavanols, flavones and catchins.

22. The pharmaceutical composition of claim 20 wherein the plant metabolite comprises flavonol.

23. The pharmaceutical composition of claim 13, wherein the polyphenol is part of a mixture of polyphenols derived from a botanical source.

24. The pharmaceutical composition of the claim 13, wherein the pharmaceutical composition further comprises a sustained release pharmaceutically acceptable carrier which controls the release of the at least one polyphonol over a desired time period within the patient's bladder.

25. The pharmaceutical composition of claim 13, wherein the composition is in a pack or kit.

26. A method for inhibiting the growth of cancer cells in the urinary bladder of a mammal having bladder cancer, wherein a composition comprising at least one polyphenol and a pharmaceutically acceptable carrier, is instilled into the bladder of the mammal having the cancer in an amount effective to inhibit the growth of cancer cells in the urinary bladder of the mammal having the bladder cancer.

27. The method of claim 26, wherein the growth of the cancer cells is inhibited by a prevention of tumor cell implantation.

28. The method of claim 26, wherein the growth of the cancer cells is inhibited by inhibition of proliferation of the cancer cells.

29. The method of claim 26, wherein the mammal is a human.

30. A sustained release preparation for placement into the bladder through the uretha comprising at least one polyphenol as a pharmaceutical active agent and a pharmaceutically acceptable carrier, wherein the carrier is capable of sustained delivery of the at least one polyphenol within the bladder.

31. The sustained release preparation of claim 26, wherein the pharmaceutical acceptable carrier is bio-erodable over a desired period of time within the bladder.

32. The sustained release preparation of claim 26 wherein the composition is in a pack or kit.

33. A kit for the treatment of bladder cancer comprising: a pharmaceutical preparation containing at least one polyphenol for installation in the bladder, at least one catheter and other appurtenances required for instillation into the bladder, and instructions for installing the catheter in the bladder and instilling the at lest one polyphenol pharmaceutical preparation.

34. A method of marketing a pharmaceutical product comprising the steps of:
including at least polyphenol as a component of the product; and
instructing a person to instill the product into a bladder through a catheter.
